(19) United States
(12) Patent Application Publication
(10) Pub. No.: US 2022/0401696 A1
(43) Pub. Date: Dec. 22, 2022

(54) STYLET FOR A PERCUTANEOUS CATHETER

(71) Applicant: TERUMO KABUSHIKI KAISHA, TOKYO (JP)

(72) Inventor: Masakazu Miyata, Kanagawa (JP)

(21) Appl. No.: 17/894,490

(22) Filed: Aug. 24, 2022

Related U.S. Application Data
(63) Continuation of application No. PCT/JP2021/006270, filed on Feb. 19, 2021.

(30) Foreign Application Priority Data
Mar. 12, 2020 (JP) 2020-043160

(51) Int. Cl. A61M 25/01; A61M 1/36; A61M 60/113; A61M 60/232; A61M 60/38

(52) U.S. Cl. CPC A61M 25/0102; A61M 1/3659; A61M 60/113; A61M 60/232; A61M 60/38; A61M 25/007

(57) ABSTRACT

A catheter system insertable into a living body to convey blood has a catheter tube and a stylet. The catheter tube has an expansion portion, a shaft portion, and a lumen. The stylet includes an outer peripheral member that extends in an axial direction and has an outer diameter the same as an inner diameter of the shaft portion, an inner peripheral member provided with an exposed portion exposed from a distal end of the outer peripheral member and provided on an inner periphery of the outer peripheral member so as to be slidable with respect to the outer peripheral member, and a fitting stopper into which a fitting member on an outer periphery of a proximal end of the outer peripheral member is fittable. A region is formed in the fitting stopper in which the outer peripheral member and the fitting member are movable.

### CROSS REFERENCE TO RELATED APPLICATIONS

This application is a continuation of PCT Application No. PCT/JP2021/006270, filed Feb. 19, 2021, based on and claiming priority to Japanese Application No. JP2020-043160, filed Mar. 12, 2020, both of which are incorporated herein by reference in their entirety.

### BACKGROUND OF THE INVENTION

The present invention relates to a stylet and a catheter system with the stylet.

Conventionally, treatment by percutaneous cardiopulmonary support (PCPS) has been performed in order to perform cardiopulmonary resuscitation, circulation assistance, and respiration assistance in emergency treatment. The percutaneous cardiopulmonary support is a method of temporarily assisting and substituting for a cardiopulmonary function using an extracorporeal circulation device.

The extracorporeal circulation device is provided with an extracorporeal circulation circuit formed of a centrifugal pump, an oxygenator, a blood removal path, a blood supply path and the like, and performs gas exchange on removed blood and supplies the blood to the blood supply path.

In a case where blood circulation is performed by the circulation circuit, the blood is circulated by a force of the pump driven by a motor. Therefore, in order to suitably perform the blood circulation, it is required to alleviate a pressure loss in a tube forming the circulation circuit.

Note that, when an inner diameter of the tube is small, the pressure loss increases, and a flow rate flowing through the circulation circuit decreases. Therefore, unless the inner diameter of the tube is set to a sufficient size, a required circulation amount of blood cannot be obtained.

In contrast, when the inner diameter of the tube is increased, an outer diameter of the tube is also increased. Therefore, when inner diameters of a blood removal catheter (tube) and a blood supply catheter (tube) inserted into a body of a patient are increased, a degree of invasion on the body of the patient increases, and a burden on the body of the patient increases.

In connection with this, for example, U.S. Patent Application Publication 2002/0010440A1 discloses a high-performance cannula capable of axially extending or contracting a cannula body (catheter) by a mandrel (stylet) to increase or decrease a diameter. According to the high-performance cannula formed in this manner, by inserting the same into a living body in a state in which the cannula body is extended in the axial direction and the diameter (outer diameter) is decreased by the mandrel, the degree of invasion on the body of the patient is decreased. Moreover, by removing the mandrel after completing the insertion of the high-performance cannula into the living body, the cannula body contracts in the axial direction to increase the diameter (inner diameter). Therefore, the pressure loss in the catheter is decreased, and a required flow rate of liquid may be secured.

In the high-performance cannula disclosed in US2002/0010440A1, when the stylet is inserted into the catheter, a distal terminal end (expansion portion) of the catheter is extended in the axial direction and the catheter is contracted radially inward. At that time, an insertion point (shaft portion) of the catheter tube which receives the stylet and which is located proximally of the catheter expansion portion might also become extended in the axial direction and likewise contracts radially inward. When the shaft portion contracts radially inward in this manner, it can contact with the stylet so that frictional resistance with the stylet increases, whereby the stylet cannot be inserted fully to a desired position, and the expansion portion does not completely contract radially inward. If this is inserted into the living body in a state in which the expansion portion does not completely contract radially inward, the degree of invasion on the body of the patient increases, which is not preferable.

### SUMMARY OF THE INVENTION

The present invention is achieved to solve the above-described problem, and an object thereof is to provide a stylet capable of suitably contracting an expansion portion radially inward when being inserted into a catheter.

A stylet that achieves the above-described object is a stylet formed to be insertable into a tube provided with an expandable expansion portion, a shaft portion provided at a proximal end of the expansion portion, and a lumen through which blood may flow, the stylet capable of extending the expansion portion in an axial direction. The stylet includes an outer peripheral member that extends in the axial direction and has an outer diameter the same as an inner diameter of the shaft portion, an inner peripheral member provided with an exposed portion exposed from a distal end of the outer peripheral member and provided on an inner periphery of the outer peripheral member so as to be slidable with respect to the outer peripheral member, a fitting member joined to an outer periphery of the outer peripheral member at a proximal end of the outer peripheral member, and a fitting stopper defining an inner region in which the outer peripheral member and the fitting member are movable over a length in the axial direction corresponding to an extension length of the expansion portion. Since the outer peripheral member has an outer diameter conformable to an inner diameter of the shaft portion and has an inner passage defined by an inner periphery of the outer peripheral member in which the inner peripheral member is received, the inner peripheral member is slidable without frictional resistance.

According to the stylet formed in the above-described manner, when the stylet is inserted into the tube, the shaft portion is about to contract radially inward, but the shaft portion comes into contact with the outer peripheral member, the contraction of the shaft portion radially inward is regulated, and the outer peripheral member does not move in the axial direction due to friction. In this state, by moving the inner peripheral member toward the distal end side with respect to the outer peripheral member, the expansion portion of the tube extends in the axial direction and suitably contracts radially inward. From above, at the time of insertion into the catheter, the expansion portion may be suitably contracted radially inward.

### BRIEF DESCRIPTION OF DRAWINGS

FIG. 1 is a system diagram illustrating an example of an extracorporeal circulation device to which a percutaneous catheter according to an embodiment of the present invention is applied.

FIG. 2 is a side view illustrating a state before a stylet according to this embodiment is inserted into a catheter.

FIG. 3 is a side sectional view illustrating the catheter.

FIG. 4 is a side view illustrating a state after the stylet according to this embodiment is inserted into the catheter.

FIG. 5A is a view for describing a braiding angle of a first reinforcing body, and FIG. 5B is a view for describing a braiding angle of a second reinforcing body.

FIG. 6 is a schematic sectional view illustrating a configuration of the stylet according to this embodiment.

FIGS. 7A to 7C are views for describing a method of using the stylet according to this embodiment.

FIG. 8 is a plan view illustrating a state before the stylet according to this embodiment is inserted into a double lumen catheter.

FIG. 9 is a side sectional view illustrating the double lumen catheter.

FIG. 10 is a plan view illustrating a state after the stylet according to this embodiment is inserted into the double lumen catheter.

FIGS. 11A to 11C are views for describing a method of using a stylet according to a variation.

FIG. 12 is a view for describing a configuration of a fitting member and a fitted portion according to the variation.

### DETAILED DESCRIPTION OF PREFERRED EMBODIMENTS

An embodiment of the present invention is hereinafter described with reference to the accompanying drawings. Note that, the following description does not limit the technical scope or meaning of terms recited in claims. Dimensional ratios in the drawings are exaggerated for convenience of description, and might be different from actual ratios.

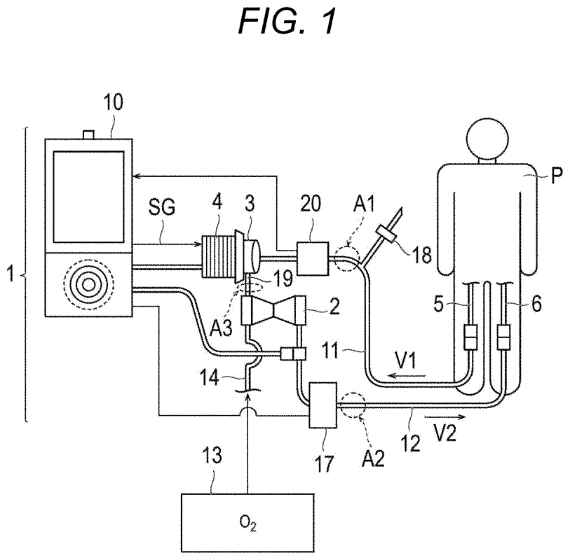

FIG. 1 is a system diagram illustrating an example of an extracorporeal circulation device to which a percutaneous catheter according to the embodiment of the present invention is applied, the extracorporeal circulation device used as percutaneous cardiopulmonary support (PCPS) that temporarily assists and substitutes for functions of the heart and lungs until a heart function recovers when the heart of a patient is weak.

According to an extracorporeal circulation device 1, it is possible to perform a veno-arterial (VA) procedure of removing blood from a vein (vena cava) of the patient by operating a pump, exchanging gas in the blood by an oxygenator to oxygenate the blood, and then returning the blood to an artery (aorta) of the patient again. The extracorporeal circulation device 1 is a device that assists the heart and lungs. Hereinafter, a procedure of removing the blood from the patient, performing predetermined extracorporeal treatment, and then supplying the blood into the body of the patient again is referred to as “extracorporeal circulation”.

As illustrated in FIG. 1, the extracorporeal circulation device 1 includes a circulation circuit that circulates the blood. The circulation circuit includes an oxygenator 2, a centrifugal pump 3, a drive motor 4 as a drive means for driving the centrifugal pump 3, a vein-side catheter (percutaneous catheter for blood removal) 5, an artery-side catheter (blood supply catheter) 6, and a controller 10 as a control unit.

The vein-side catheter (blood removal catheter) 5 is inserted from the femoral vein, and a distal end of the vein-side catheter 5 is indwelled in the right atrium via the inferior vena cava. The vein-side catheter 5 is connected to the centrifugal pump 3 via a blood removal tube (blood removal line) 11. The blood removal tube 11 is a pipeline that sends the blood.

The artery-side catheter (blood supply catheter) 6 is inserted from the femoral artery.

When the drive motor 4 operates the centrifugal pump 3 by a command SG of the controller 10, the centrifugal pump 3 may remove the blood from the blood removal tube 11 and pass the blood via the oxygenator 2, then return the blood to a patient P through a blood supply tube (blood supply catheter) 12.

The oxygenator 2 is arranged between the centrifugal pump 3 and the blood supply tube 12. The oxygenator 2 performs gas exchange (oxygenation and/or carbon dioxide removal) on the blood. The oxygenator 2 is, for example, a membrane oxygenator, and a hollow fiber membrane oxygenator is especially preferably used. Oxygen gas is supplied from an oxygen gas supply unit 13 to the oxygenator 2 via a tube 14. The blood supply tube 12 is a pipeline that connects the oxygenator 2 to the artery-side catheter 6.

As the blood removal tube 11 and the blood supply tube 12, for example, a pipeline made of an elastically deformable and flexible synthetic resin having high transparency such as a vinyl chloride resin or silicone rubber may be used. In the blood removal tube 11, the blood as liquid flows in a V1 direction, and in the blood supply tube 12, the blood flows in a V2 direction.

In the circulation circuit illustrated in FIG. 1, an ultrasonic bubble detection sensor 20 is arranged in the middle of the blood removal tube 11. A fast clamp 17 is arranged in the middle of the blood supply tube 12.

In a case where a bubble is mixed in the circulation circuit with the blood due to an erroneous operation of a three-way stopcock 18 or a breakage of the tube and the like during the extracorporeal circulation, then the ultrasonic bubble detection sensor 20 detects the mixed bubble. In a case where the ultrasonic bubble detection sensor 20 detects that there is the bubble in the blood sent in the blood removal tube 11, the ultrasonic bubble detection sensor 20 transmits a detection signal to the controller 10. On the basis of this detection signal, the controller 10 performs notification of a warning by an alarm, and decreases a rotation speed of the centrifugal pump 3 or stops the centrifugal pump 3. The controller 10 further instructs the fast clamp 17 to immediately close the blood supply tube 12 by the fast clamp 17. This prevents the bubble from being sent into the body of the patient P. The controller 10 controls an operation of the extracorporeal circulation device 1 to prevent mixing of the bubble into the body of the patient P.

The tube 11 (tube 12, 19) of the circulation circuit of the extracorporeal circulation device 1 is provided with a pressure sensor. For example, the pressure sensor may be attached to any one or all of an attachment position A1 of the blood removal tube 11, an attachment position A2 of the blood supply tube 12 of the circulation circuit, and an attachment position A3 of a connection tube 19 that connects the centrifugal pump 3 to the oxygenator 2. As a result, a pressure in the tube 11 (12, 19) may be measured by the pressure sensor when the extracorporeal circulation is performed on the patient P by the extracorporeal circulation device 1. Note that, the attachment position of the pressure sensor is not limited to the attachment positions A1, A2, and A3 described above, and may be any position of the circulation circuit.

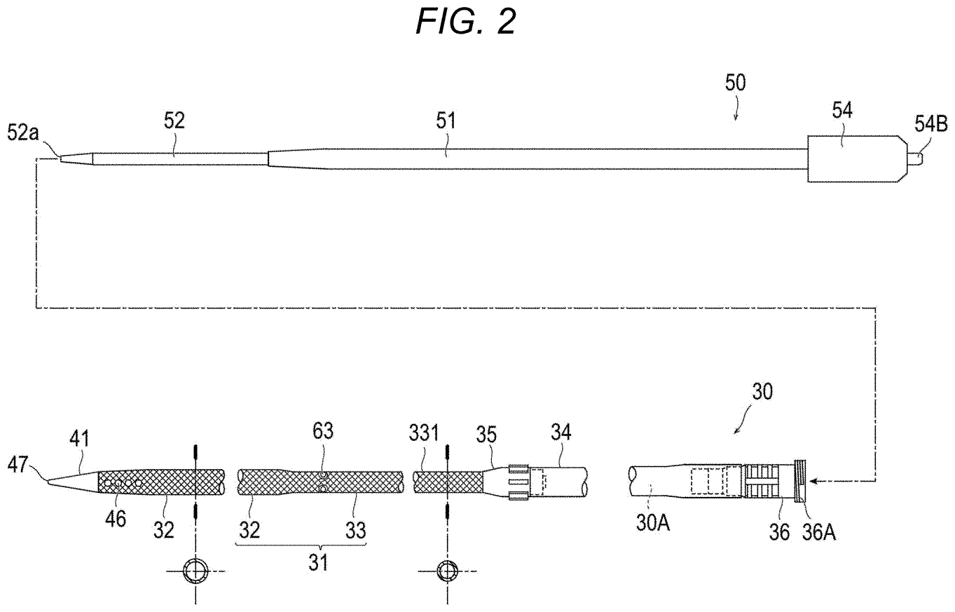

Next, a configuration of a percutaneous catheter (hereinafter, referred to as a “catheter”) 30 into which a stylet 50 according to the embodiment of the present invention is inserted is described with reference to FIGS. 2 to 5B. FIGS. 2 to 5B are views for describing the configuration of the catheter 30. The catheter 30 is used as the vein-side catheter (blood removal catheter) 5 in FIG. 1. Note that, the configuration of the catheter 30 described below is an example, and the configuration of the catheter into which the stylet 50 according to this embodiment is inserted is not limited to the following configuration.

As illustrated in FIG. 2, the catheter 30 includes a catheter tube 31 provided with a first side hole 63 and a second side hole 46, a distal end tip 41 arranged at a distal end of the catheter tube 31 and provided with a through-hole 47, a clamping tube 34 arranged on a proximal end side of the catheter tube 31, a catheter connector 35 that connects the catheter tube 31 to the clamping tube 34, and a lock connector 36.

Note that, in this specification, a side to be inserted into a living body is referred to as a “distal end” or a “distal end side”, and a hand side operated by an operator is referred to as a “proximal end” or a “proximal end side”. A distal end part means a certain range including the distal end (most distal end) and its periphery, and a proximal end part means a certain range including the proximal end (most proximal end) and its periphery.

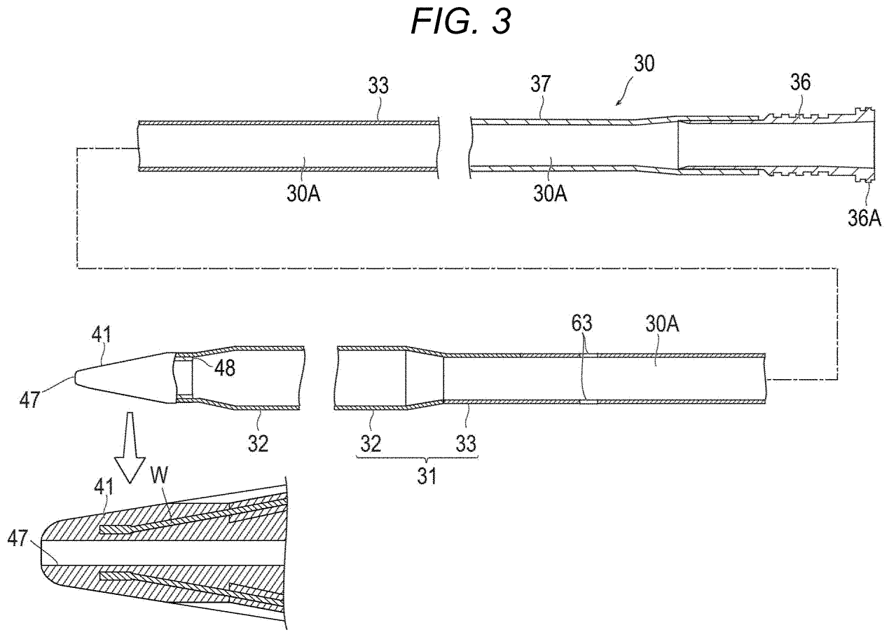

As illustrated in FIG. 3, the catheter 30 includes a lumen 30A penetrating from a distal end to a proximal end thereof. The through-hole 47 provided on the distal end tip 41, and the first and second side holes 63 and 46 provided on the catheter tube 31 are arranged in different blood removal targets in the living body such that the blood may be efficiently removed.

When the catheter 30 is inserted into the living body, the stylet 50 illustrated in FIG. 2 is used. The stylet 50 is inserted into the lumen 30A of the catheter 30, and the catheter 30 and the stylet 50 are inserted together into the living body in a state of being integrated in advance.

Hereinafter, each configuration of the catheter 30 is described. Note that, the configuration of the catheter 30 is not limited to the following.

As illustrated in FIG. 2, the catheter tube 31 includes an expansion portion 32 and a shaft portion 33 connected to a proximal end side of the expansion portion 32.

The expansion portion 32 is formed to have higher elasticity than that of the shaft portion 33. The expansion portion 32 is formed to have larger outer and inner diameters than those of the shaft portion 33.

Lengths of the expansion portion 32 and the shaft portion 33 are set to lengths necessary for arranging the through-hole 47 of the distal end tip 41 and the first and second side holes 63 and 46 of the catheter tube 31 in desired blood removal targets. The length of the expansion portion 32 may be set to, for example, 20 to 40 cm, and the length of the shaft portion 33 may be set to, for example, 20 to 30 cm.

In this embodiment, the blood removal targets are two sites: the right atrium and the inferior vena cava. The catheter 30 is inserted into the living body to be indwelled there such that the through-hole 47 of the distal end tip 41 and the second side hole 46 of the catheter tube 31 are arranged in the right atrium, and the first side hole 63 of the catheter tube 31 is arranged in the inferior vena cava.

In a state in which the through-hole 47, the second side hole 46, and the first side hole 63 are arranged in the blood removal targets, the expansion portion 32 is arranged in the inferior vena cava, which is a relatively large blood vessel, and the shaft portion 33 is arranged in the femoral vein, which is a relatively small blood vessel.

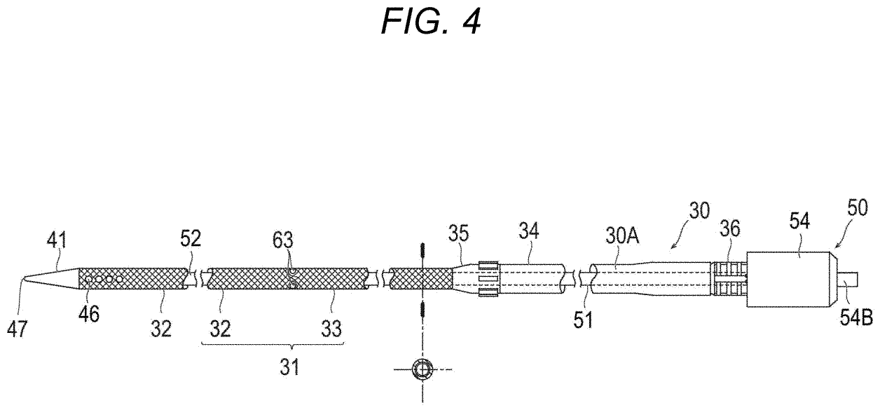

When the stylet 50 is inserted into the lumen 30A of the catheter 30, the expansion portion 32 having high elasticity extends in an axial direction and the outer and inner diameters thereof decrease as illustrated in FIG. 4. At that time, the outer diameter of the expansion portion 32 is substantially the same as the outer diameter of the shaft portion 33. Since the catheter 30 is inserted into the living body in a state in which the expansion portion 32 is extended in the axial direction and the outer and inner diameters thereof decrease, the catheter 30 may be inserted in a minimally invasive manner.

When the stylet 50 is removed from the lumen 30A of the catheter 30 after the catheter 30 is indwelled in the living body, the expansion portion 32 contracts from the state of being extended in the axial direction so that it will return to a larger inner diameter. Herein, the expansion portion 32 is arranged in the inferior vena cava, which is a relatively large blood vessel. Therefore, the outer diameter of the expansion portion 32 may be increased, and accordingly, the inner diameter may be increased.

Herein, a pressure loss in the expansion portion 32 is obtained by a total length of the expansion portion 32×(average) passage cross-sectional area. That is, by increasing the inner diameter of the expansion portion 32, the pressure loss in the expansion portion 32 is decreased. When the pressure loss in the expansion portion 32 is decreased, a flow rate of the blood flowing through the circulation circuit increases. Therefore, in order to obtain a sufficient blood circulation amount, it is necessary to increase the inner diameter of the expansion portion 32.

In contrast, in a case where a wall thickness is substantially constant, when the inner diameters of the expansion portion 32 and the shaft portion 33 are increased, the outer diameters increase, so that a burden on the patient increases when the catheter 30 is inserted into the living body, which hinders a minimally invasive procedure.

From the above-described viewpoint, the inner diameter of the expansion portion 32 may be set to, for example, 9 to 11 mm, and the inner diameter of the shaft portion 33 may be set to, for example, 4 to 8 mm. The wall thickness of each of the expansion portion 32 and the shaft portion 33 may be set to, for example, 0.4 to 0.5 mm.

As illustrated in FIG. 2, it is preferable that a distal end part of the expansion portion 32 forms a tapered portion that gradually becomes thinner from the center of the expansion portion 32 toward a distal end side in the axial direction. As a result, the inner diameter of a distal end of the expansion portion 32 is continuous to an inner diameter of the distal end tip 41 arranged on the distal end side thereof.

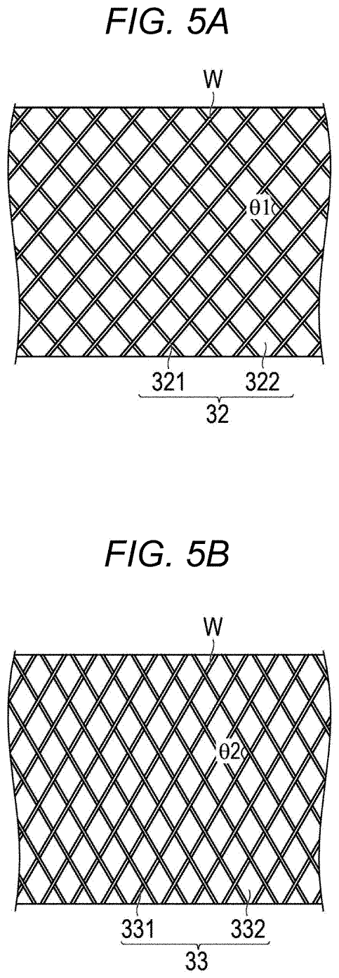

As illustrated in FIG. 5A, the expansion portion 32 includes a first reinforcing body 321 including wires W braided so as to cross each other, and a first resin layer 322 provided so as to cover the first reinforcing body 321.

As illustrated in FIG. 5B, the shaft portion 33 includes a second reinforcing body 331 including wires W braided so as to cross each other, and a second resin layer 332 provided so as to cover the second reinforcing body 331.

As illustrated in FIG. 5A, the first reinforcing body 321 is formed by braiding the wires W to form a braiding angle θ1. As illustrated in FIG. 5B, the second reinforcing body 331 is formed by braiding the wires W to form a braiding angle 82.

In this specification, the braiding angles θ1 and θ2 are defined as inner angles in the axial direction out of the angles formed by the crossing wires W, as illustrated in FIGS. 5A and 5B.

As illustrated in FIGS. 5A and 5B, the braiding angle θ1 of the first reinforcing body 321 is made smaller than the braiding angle θ2 of the second reinforcing body 331. Therefore, an inclination angle of the wire W forming the first reinforcing body 321 with respect to the axial direction is smaller than that in a case where the braiding angle of the first reinforcing body 321 is larger than the braiding angle of the second reinforcing body 331. Note that, the braiding angle θ1 of the first reinforcing body 321 may be made larger than the braiding angle θ2 of the second reinforcing body 331.

Herein, as the expansion portion 32 extends in the axial direction, the wire W forming the first reinforcing body 321 of the expansion portion 32 is deformed such that the inclination angle with respect to the axial direction gradually decreases. When the inclination angle of the wire W forming the first reinforcing body 321 of the expansion portion 32 with respect to the axial direction becomes approximately 0, the extension of the expansion portion 32 in the axial direction is regulated.

Therefore, by making the braiding angle θ1 of the first reinforcing body 321 smaller than the braiding angle θ2 of the second reinforcing body 331, as compared with a case where the braiding angle of the first reinforcing body 321 is larger than the braiding angle of the second reinforcing body 331, an extension distance in the axial direction of the expansion portion 32 accompanying the insertion of the stylet 50 into the catheter 30 is shortened.

The braiding angle θ1 of the first reinforcing body 321 is not especially limited, but is 100 to 120 degrees. The braiding angle θ2 of the second reinforcing body 331 is not especially limited, but is 130 to 150 degrees. By making the braiding angle θ2 of the second reinforcing body 331 larger than the braiding angle θ1 of the first reinforcing body 321 in this manner, kink resistance of the second reinforcing body 331 may be improved. Therefore, the catheter 30 may be suitably inserted into the living body in the femoral vein having a complicated configuration.

As illustrated in FIGS. 5A and 5B, the first reinforcing body 321 of the expansion portion 32 is braided sparser than the second reinforcing body 331 of the shaft portion 33. According to this configuration, the expansion portion 32 may be made softer than the shaft portion 33, and elasticity may be enhanced.

In this embodiment, the wire W is formed of a well-known shape memory material of shape memory metal or a shape memory resin. As the shape memory metal, for example, a titanium-based (Ni—Ti, Ti—Pd, Ti—Nb—Sn and the like) or copper-based alloy may be used. As the shape memory resin, for example, an acrylic resin, a transisoprene polymer, polynorbornene, a styrene-butadiene copolymer, and polyurethane may be used.

Since the wire W is formed of the shape memory material, a contraction distance in the axial direction of the expansion portion 32 accompanying the removal of the stylet 50 from the catheter 30 is the same as the extension distance in the axial direction of the expansion portion 32 accompanying the insertion of the stylet 50 into the catheter 30.

A diameter of the wire W is preferably 0.1 to 0.2 mm.

By setting the diameter of the wire W to 0.1 mm or larger, a function as the reinforcing body for improving strength may be suitably exhibited.

In contrast, by setting the diameter of the wire W to 0.2 mm or smaller, the inner diameter of the expansion portion 32 may be increased while decreasing the outer diameter thereof, so that it is possible to achieve both suppression in burden on the body of the patient at the time of insertion of the catheter 30 and decrease in pressure loss. In this embodiment, a cross section of the wire W is circular, but is not limited thereto, and may be rectangular, square, elliptical and the like.

The first resin layer 322 of the expansion portion 32 is formed of a soft material having hardness lower than that of the second resin layer 332 of the shaft portion 33. According to this configuration, the expansion portion 32 may be made softer than the shaft portion 33, and elasticity may be enhanced.

The first and second resin layers 322 and 332 may be formed using vinyl chloride, silicon, polyethylene, nylon, urethane, polyurethane, a fluororesin, a thermoplastic elastomer resin and the like, or a composite material thereof.

The silicon material has high biocompatibility, and the material itself is soft, so that this has an advantage that this does not easily damage a blood vessel. The polyethylene material is soft and has hardness to withstand a pressure. Moreover, the polyethylene material has biocompatibility comparable to that of the silicon material. The polyethylene material is harder than silicon, and has an advantage that this is easily inserted into a thin blood vessel. The polyurethane material has an advantage that this becomes soft after insertion. As the materials of the first and second resin layers 322 and 332, applicable materials may be used by using the advantages of these materials.

A hydrophilic coating may be applied to the polyurethane material. In this case, since a tube surface is smooth, this may be easily inserted into the blood vessel, and the blood vessel wall is less likely to be damaged. The blood and proteins are less likely to adhere, and it may be expected that thrombus formation is prevented.

A method of forming the expansion portion 32 and the shaft portion 33 is not especially limited, but they may be formed by, for example, dip coating (immersion method), insert molding and the like. Note that, it is sufficient that at least outer surfaces of the reinforcing bodies 321 and 331 are covered with the resin layers 322 and 332, respectively.

As illustrated in FIG. 2, the expansion portion 32 includes the second side hole 46. As illustrated in FIG. 2, a plurality of (four in FIG. 2) second side holes 46 is provided in the axial direction. A plurality of second side holes 46 is preferably provided also in a circumferential direction. The second side hole 46 serves as a blood removal hole.

As illustrated in FIG. 2, the shaft portion 33 includes the first side hole 63. The first side hole 63 serves as a blood removal hole. A plurality of first side holes 63 is preferably provided in a circumferential direction. In this embodiment, the shaft portion 33 is provided with four first side holes 63 in the circumferential direction. As a result, even if one first side hole 63 is adsorbed to the blood vessel wall and blocked by the blood removal, the blood removal may be performed by another first side hole 63, so that blood circulation may be stably performed.

As illustrated in FIGS. 2 to 4, the distal end tip 41 is arranged at the distal end of the expansion portion 32. The distal end tip 41 has a tapered shape a diameter of which is gradually decreased toward the distal end side.

A flat receiving surface 48 that abuts a flat surface 52a of the stylet 50 used before the catheter 30 is inserted into the living body is formed inside the distal end tip 41.

As illustrated in FIG. 3, the distal end tip 41 is formed to house a distal end of the wire W. The distal end tip 41 includes the through-hole 47. The through-hole 47 serves as a blood removal hole. The through-hole 47 of the distal end tip 41 forms a part of the lumen 30A of the catheter 30. The distal end tip 41 may be formed of, for example, urethane.

By fixing a hard distal end tip 41 to the distal end part of the expansion portion 32, it is possible to effectively prevent the expansion portion 32 from being crushed at the time of blood removal.

As illustrated in FIGS. 2 to 4, the clamping tube 34 is provided on a proximal end side of the shaft portion 33. A lumen into which the stylet 50 may be inserted is provided inside the clamping tube 34. The clamping tube 34 may be formed using a material similar to that of the catheter tube 31.

As illustrated in FIGS. 2 and 4, the catheter connector 35 connects the shaft portion 33 to the clamping tube 34. A lumen into which the stylet 50 may be inserted is provided inside the catheter connector 35.

As illustrated in FIGS. 2 to 4, the lock connector 36 is connected to a proximal end side of the clamping tube 34. A lumen into which the stylet 50 may be inserted is provided inside the lock connector 36. A male screw portion 36A provided with a screw thread is provided on an outer surface on a proximal end side of the lock connector 36.

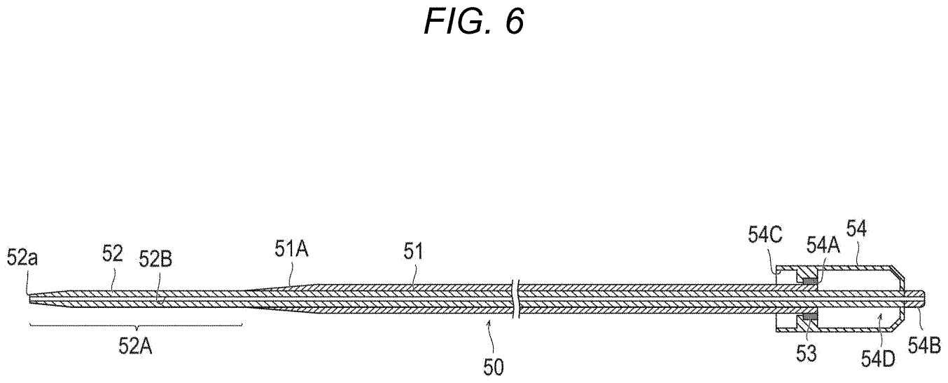

Next, a configuration of the stylet 50 according to this embodiment is described with reference to FIG. 6 and the like. FIG. 6 is a view for describing the configuration of the stylet 50 according to this embodiment.

As illustrated in FIG. 6, the stylet 50 includes an outer peripheral member 51 that extends in the axial direction, an inner peripheral member 52 provided on an inner periphery of the outer peripheral member 51, a fitting member 53 joined to an outer periphery of a proximal end of the outer peripheral member 51, and a fitting stopper 54 into which the fitting member 53 may be fitted.

An outer diameter of the outer peripheral member 51 is formed so as to be the same as the inner diameter of the shaft portion 33. Note that, the fact that this is the same as the inner diameter of the shaft portion 33 not only indicates that they are completely the same but also includes some tolerance error.

As illustrated in FIG. 6, a distal end part 51A of the outer peripheral member 51 is tapered such that the outer diameter gradually decreases toward a distal end from the outer peripheral member 51 toward the inner peripheral member 52. According to this configuration, since the outer periphery of the distal end part 51A of the outer peripheral member 51 has a gentle tapered shape, the catheter 30 has a smooth transitional shape without a step following the shape of the distal end part 51A in a state in which the stylet 50 is inserted into and integrated with the catheter 30. Therefore, insertability of the catheter 30 into the living body is improved.

An inner diameter of the outer peripheral member 51 is formed to be slightly larger than an outer diameter of the inner peripheral member 52. Therefore, the inner peripheral member 52 is slidable in the axial direction (right-left direction in FIG. 6) with respect to the outer peripheral member 51 within an inner passage of outer peripheral member 51.

The outer peripheral member 51 is an elongated body having relatively high rigidity. A material forming the outer peripheral member 51 is not especially limited, but the material similar to that of the first and second resin layers 322 and 332 described above may be used.

The inner peripheral member 52 is provided on the inner periphery of the outer peripheral member 51 so as to be slidable with respect to the outer peripheral member 51. As illustrated in FIG. 6, the inner peripheral member 52 includes an exposed portion 52A exposed from the distal end of the outer peripheral member 51.

A length in the axial direction of the exposed portion 52A is preferably the same as or shorter than the length in the axial direction of the expansion portion 32 before being extended. According to this configuration, the expansion portion 32 may be suitably contracted radially inward.

An entire length in the axial direction of the inner peripheral member 52 is formed to be longer than an entire length in the axial direction of the catheter 30 before the expansion portion 32 is extended. In other words, the entire length in the axial direction of the inner peripheral member 52 is formed to be the same as the entire length in the axial direction of the catheter 30 after the expansion portion 32 becomes extended.

The inner peripheral member 52 is provided with a guidewire lumen 52B into which a guidewire (not illustrated) may be inserted. The outer peripheral member 51 and the inner peripheral member 52 are guided by the guide wire to be inserted into the living body together with the catheter 30.

As illustrated in FIG. 2, a distal end of the inner peripheral member 52 is provided with the flat surface 52a that the receiving surface of the distal end tip 41 abuts when inserted. The inner peripheral member 52 is formed such that the outer diameter and an inner diameter are uniform in the axial direction.

The inner peripheral member 52 is an elongated body having relatively high rigidity. The inner peripheral member 52 is preferably formed of a material softer than that of the outer peripheral member 51. A material forming the inner peripheral member 52 is not especially limited, but the material similar to that of the first and second resin layers 322 and 332 described above may be used. According to this configuration, it is possible to relatively increase rigidity of a proximal end of the stylet 50 while softening a distal end of the stylet 50. Therefore, when the stylet 50 and the catheter 30 are inserted into the living body, it is possible to prevent living tissue from being damaged, and stiffness that enables transmission of a pushing force to the distal end side by a hand side operation to the distal end tip 41 is provided.

As illustrated in FIG. 6, the fitting member 53 is joined to the outer periphery of the proximal end of the outer peripheral member 51. A method of joining the fitting member 53 to the outer peripheral member 51 is not especially limited, and is, for example, adhesion using an adhesive.

The fitting member 53 is formed of, for example, rubber, and has elasticity. Note that, a material forming the fitting member 53 is not limited to rubber, and may be any material that may be strongly fitted into a fitted portion 54A. The fitting member 53 is formed to be fittable into the fitted portion 54A of the fitting stopper 54 to be described later. That is, an outer diameter of the fitting member 53 is formed to be slightly larger than an inner diameter of the fitted portion 54A of the fitting stopper 54.

As illustrated in FIG. 6, the fitting stopper 54 is provided at the proximal end of the stylet 50. As illustrated in FIG. 6, the fitting stopper 54 includes the fitted portion 54A, a hub 54B, and a screw ring 54C.

The fitted portion 54A is provided on a proximal end side of the screw ring 54C. The fitted portion 54A is a lumen having the inner diameter smaller than the outer diameter of the fitting member 53.

The hub 54B is provided at a proximal end of the fitting stopper 54 so as to be grippable. After the catheter 30 is indwelled in the living body, the stylet 50 is removed from the catheter 30 by pulling out the hub 54B to the proximal end side.

The screw ring 54C includes a female screw portion (not illustrated) provided with a screw groove on an inner surface of an inner cavity. The stylet 50 may be attached to the catheter 30 by screwing the female screw portion of the screw ring 54C into the male screw portion 36A of the lock connector 36.

Moreover, the fitting stopper 54 includes a region 54D in which the outer peripheral member 51 and the fitting member 53 are movable between the fitted portion 54A and the hub 54B. A length in the axial direction of the region 54D is made the same as an extension length of the remaining expansion portion 32 when the shaft portion 33 starts contracting. According to this configuration, the expansion portion 23 may be suitably contracted radially inward.

<Method of Using Stylet>

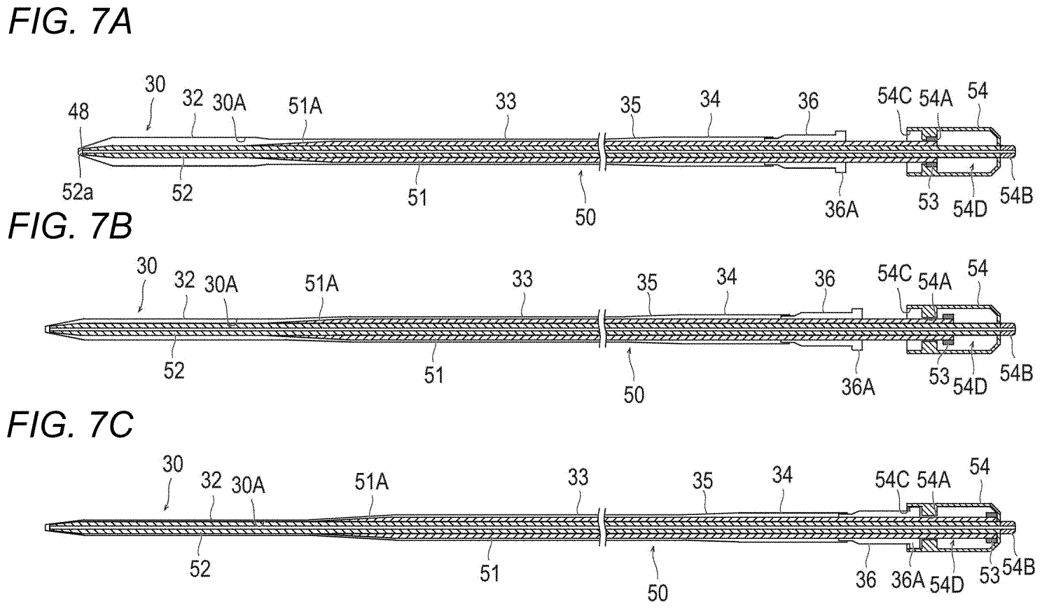

Next, a method of using the above-described stylet 50 is described with reference to FIGS. 7A to 7C. FIGS. 7A to 7C are views for describing the method of using the stylet 50 according to this embodiment.

First, as illustrated in FIG. 7A, the stylet 50 is inserted into the lumen 30A of the catheter 30 in preparation for insertion into a living body. The stylet 50 sequentially passes through the inside of the shaft portion 33 and the expansion portion 32, until the flat surface 52a of the inner peripheral member 52 of the stylet 50 abuts the receiving surface 48 of the distal end tip 41.

Herein, the entire length in the axial direction of the outer peripheral member 51 and the inner peripheral member 52 is made longer than the entire length in the axial direction of the catheter 30 before the expansion portion 32 becomes extended as illustrated in FIG. 2. Therefore, the expansion portion 32 is pressed toward the distal end side in a state in which the flat surface 52a of the inner peripheral member 52 of the stylet 50 abuts the receiving surface 48 of the distal end tip 41.

Then, the distal end of the expansion portion 32 is pulled toward the distal end side as illustrated in FIG. 7B. As a result, the catheter 30 receives a force to extend in the axial direction, and the expansion portion 32 having relatively high elasticity out of the catheter 30 extends (stretches) in the axial direction. The shaft portion 33 would also tend to contract radially inward, but the shaft portion 33 comes into contact with the outer peripheral member 51 so that contraction of the shaft portion 33 radially inward is regulated, and the outer peripheral member 51 does not move in the axial direction due to friction. In this state, by moving the inner peripheral member 52 toward the distal end side with respect to the outer peripheral member 51, the expansion portion 32 extends in the axial direction and suitably contracts radially inward without being hindered by the friction. At that time of relative movement between outer peripheral member 51 and inner peripheral member 52, the fitting member 53 fitted into the fitted portion 54A of the fitting stopper 54 is displaced from the fitted portion 54A.

Thereafter, the stylet 50 is attached to the catheter 30 by screwing the female screw portion of the screw ring 54C into the male screw portion 36A provided on the lock connector 36 of the catheter as illustrated in FIG. 7C. At that time, as illustrated in FIG. 7C, the proximal ends of the fitting member 53 and the outer peripheral member 51 abut a proximal end of the region 54D of the fitting stopper 54.

Next, the catheter 30 into which the stylet 50 is inserted is inserted along the guide wire (not illustrated) inserted in advance into a target site in the living body. At that time, since the stylet 50 is inserted into the catheter 30, the outer diameter of the expansion portion 32 is substantially the same as the outer diameter of the shaft portion 33, and the catheter 30 may be inserted into the living body in a minimally invasive manner, and the burden on the body of the patient may be suppressed.

The catheter 30 is inserted into the living body until the through-hole 47 of the distal end tip 41 and the second side hole 46 of the catheter tube 31 are arranged in the right atrium and the first side hole 63 of the catheter tube 31 is arranged in the inferior vena cava to be indwelled there. In a state in which the through-hole 47, the first side hole 63, and the second side hole 46 are arranged in the blood removal targets, the expansion portion 32 is arranged in the inferior vena cava, which is a relatively large blood vessel, and the shaft portion 33 is arranged in the femoral vein, which is a relatively small blood vessel.

Next, the stylet 50 and the guide wire are removed from the catheter 30. At that time, the stylet 50 and the guide wire are temporarily pulled out to a site of the clamping tube 34 of the catheter 30 and clamped by forceps (not illustrated), and then completely removed from the catheter 30. When the stylet 50 is removed from the lumen of the catheter 30, the catheter 30 is released from the force to axially extend that the catheter 30 receives from the stylet 50. Therefore, the expansion portion 32 contracts in the axial direction, and the inner diameter of the expansion portion 32 increases. As a result, the pressure loss in the expansion portion 32 may be decreased, and a required flow rate of liquid may be secured.

Next, the lock connector 36 of the catheter 30 is connected to the blood removal tube 11 of the extracorporeal circulation device in FIG. 1. After confirming that the connection of the catheter on the blood supply side is completed, the forceps of the clamping tube 34 are released to start the extracorporeal circulation.

When the extracorporeal circulation ends, the catheter 30 is removed from the blood vessel and hemostatic repair is performed by a surgical procedure as necessary at an insertion site.

As described above, the stylet 50 according to this embodiment is the stylet 50 formed to be insertable into the catheter 30 provided with the expandable expansion portion 32, the shaft portion 33 provided at the proximal end of the expansion portion 32, and the lumen 30A through which the blood may flow, the stylet 50 capable of extending the expansion portion 32 in the axial direction. The stylet 50 includes the outer peripheral member 51 that extends in the axial direction and has the same outer diameter as the inner diameter of the shaft portion 33, the inner peripheral member 52 provided with the exposed portion 52A exposed from the distal end of the outer peripheral member 51 and is provided on the inner periphery of the outer peripheral member 51 so as to be slidable with respect to the outer peripheral member 51, the fitted portion 54A into which the fitting member 53 joined to the outer periphery of the proximal end of the outer peripheral member 51 is fittable, and the fitting stopper 54 in which the region 54D in which the outer peripheral member 51 and the fitting member 53 may move is formed. According to the stylet 50 formed in this manner, when the stylet 50 is inserted into the catheter 30, the shaft portion 33 is about to contract radially inward, but the shaft portion 33 comes into contact with the outer peripheral member 51, the contraction of the shaft portion 33 radially inward is regulated, and the outer peripheral member 51 does not move in the axial direction due to friction. In this state, by moving the inner peripheral member 52 toward the distal end side with respect to the outer peripheral member 51, the expansion portion 32 of the catheter 30 extends in the axial direction and suitably contracts radially inward. As a result of the above actions, at the time of insertion into the catheter 30, the expansion portion 32 may be suitably contracted radially inward.

<Variation of Catheter>

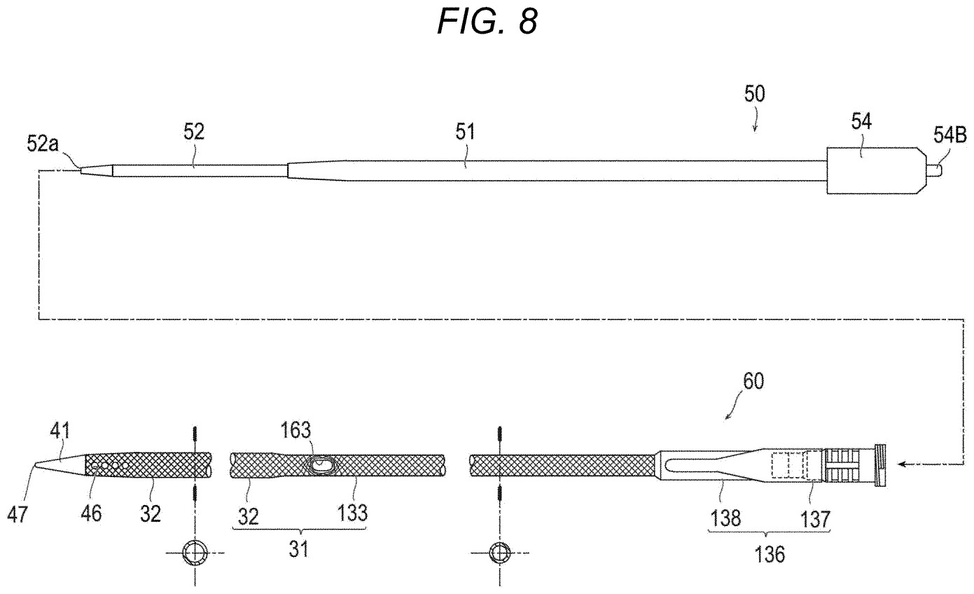

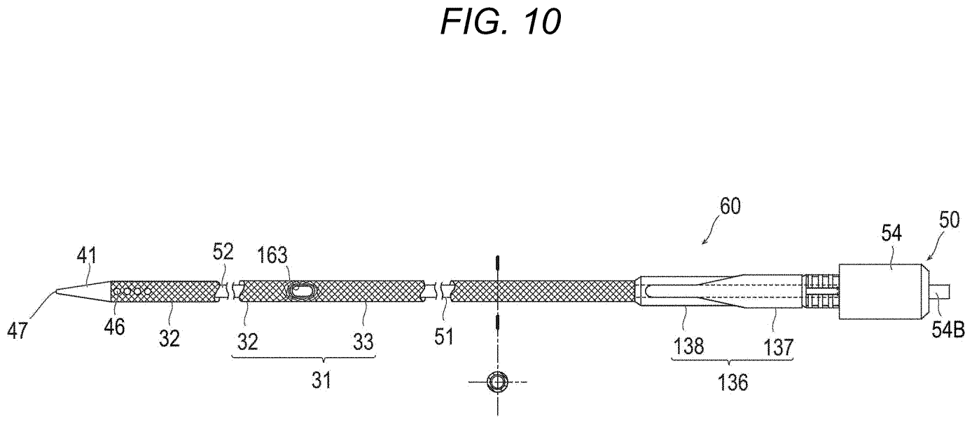

Next, a variation of the catheter is described. In the embodiment described above, the stylet 50 is applied to the catheter 30 provided with one lumen 30A. However, this may also be used for a catheter 60 provided with a double lumen as illustrated in FIGS. 8 to 10. Hereinafter, a configuration of the catheter 60 provided with the double lumen is described with reference to FIGS. 8 to 10.

The catheter 60 is a so-called double lumen catheter, and may simultaneously perform both blood supply and blood removal. Therefore, in this embodiment, a procedure is performed using only one catheter 60 without using two catheters of a vein-side catheter (blood removal catheter) 5 and an artery-side catheter (blood supply catheter) 6 in the extracorporeal circulation device in FIG. 1.

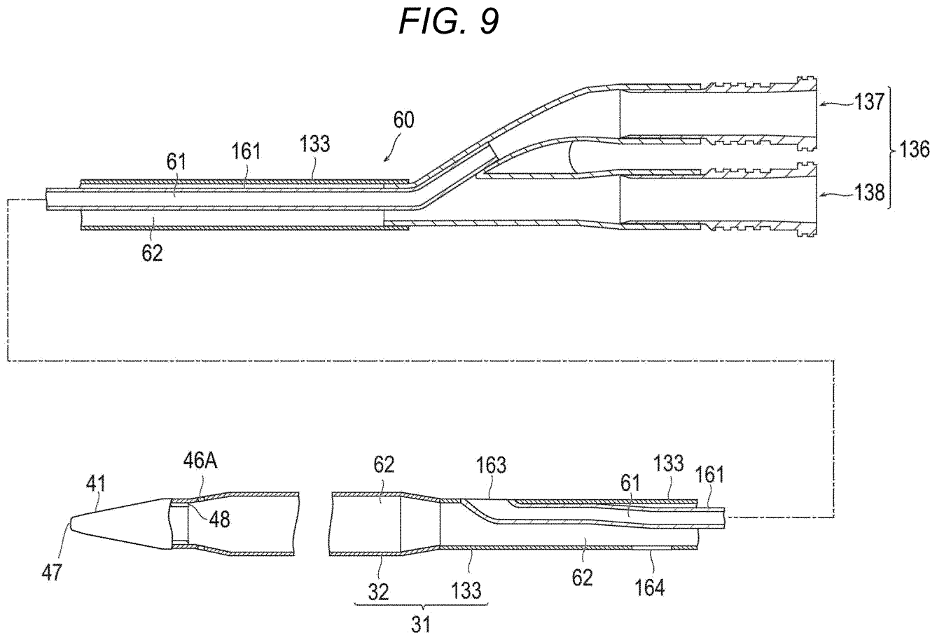

As illustrated in FIGS. 8 and 9, the catheter 60 has a double tube structure in which a third tube 161 provided with a first lumen 61 communicating with a blood supply side hole 163 is arranged in an inner cavity of a shaft portion 133.

According to the catheter 60, it is possible to perform veno-venous (VV) oxygenator extracorporeal blood circulation of removing blood from a vein (vena cava) of a patient by operating a pump of the extracorporeal circulation device, exchanging gas in the blood by an oxygenator to oxygenate the blood, and then returning the blood to an artery (aorta) of the patient again.

As illustrated in FIGS. 8 to 10, the catheter 60 includes an expansion portion 32, a shaft portion 133, a distal end tip 41 arranged at a distal end of the expansion portion 32, and the third tube 161 arranged in an inner cavity of the shaft portion 133. Since configurations of the expansion portion 32 and the distal end tip 41 are the same as those of the catheter 30 of the first embodiment, the description thereof is omitted.

As illustrated in FIG. 9, the catheter 60 includes a first lumen 61 serving as a blood supply path and a second lumen 62 serving as a blood removal path.

The first lumen 61 is formed in an inner cavity of the third tube 161. The second lumen 62 is formed in the inner cavity of the expansion portion 32 and the shaft portion 133, and penetrates from a distal end to a proximal end.

The shaft portion 133 is provided with the blood supply side hole 163 communicating with the first lumen 61, which is the blood supply path.

The shaft portion 133 is provided with a blood removal side hole 164 communicating with the second lumen 62, which is the blood removal path.

Each of the blood supply side hole 163 and the blood removal side hole 164 is formed into an elliptical shape.

The third tube 161 is inserted into the second lumen 62 from a proximal end side of the shaft portion 133 and connected to the blood supply side hole 163.

The blood supply side hole 163 is arranged in a blood supply target in a living body, and the blood oxygenated by the oxygenator is delivered into the living body via the blood supply side hole 163.

A through-hole 47 provided on the distal end tip 41, a second side hole 46 provided on the expansion portion 32, and the blood removal side hole 164 provided on the shaft portion 133 are arranged in different blood removal targets in the living body such that the blood may be efficiently removed. Even if the through-hole 47, the second side hole 46, or the blood removal side hole 164 is adsorbed to a blood vessel wall and blocked, the blood removal may be performed by the hole that is not blocked, so that extracorporeal circulation may be stably performed.

In this embodiment, the catheter 60 is inserted from the internal jugular vein of the neck, and a distal end thereof is indwelled in the inferior vena cava via the superior vena cava and the right atrium. The blood supply target is the right atrium and the blood removal target is two sites: the superior vena cava and the inferior vena cava.

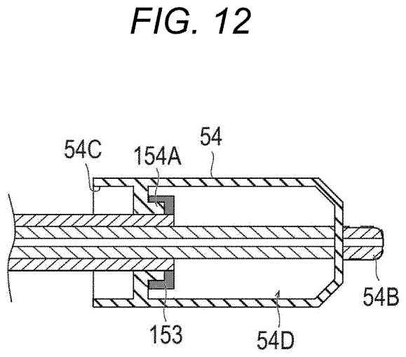

The catheter 60 is inserted into the living body to be indwelled there such that the through-hole 47 of the distal end tip 41 and the second side hole 46 of the expansion portion 32 are arranged in the inferior vena cava, and the blood removal side hole 164 of the shaft portion 133 is arranged in the internal jugular vein in a state in which the stylet 50 is inserted as illustrated in FIG. 12.

The expansion portion 32 is formed to have a larger inner diameter than that of the shaft portion 133. In a state in which the through-hole 47, the second side hole 46, and the blood removal side hole 164 are arranged in the blood removal targets, the expansion portion 32 is arranged in the inferior vena cava, which is a relatively large blood vessel, and the shaft portion 133 is arranged in the femoral vein, which is a relatively small blood vessel.

As illustrated in FIG. 9, a lock connector 136 includes a first lock connector 137 communicating with the first lumen 61 and a second lock connector 138 provided in parallel with the first lock connector 137 and communicating with the second lumen 62. The lock connector 136 is a Y-shaped Y connector formed by branching the first lock connector 137 from the second lock connector 138.

The first lock connector 137 is connected to a proximal end part of the third tube 161. The second lock connector 138 is coaxially connected to a proximal end part of the shaft portion 133. A blood supply tube (blood supply line) is connected to the first lock connector 137, and a blood removal tube (blood removal line) is connected to the second lock connector 138.

As described above, according to the catheter 60 according to this embodiment, one catheter may perform both functions of blood removal and blood supply.

<Variation of Stylet>

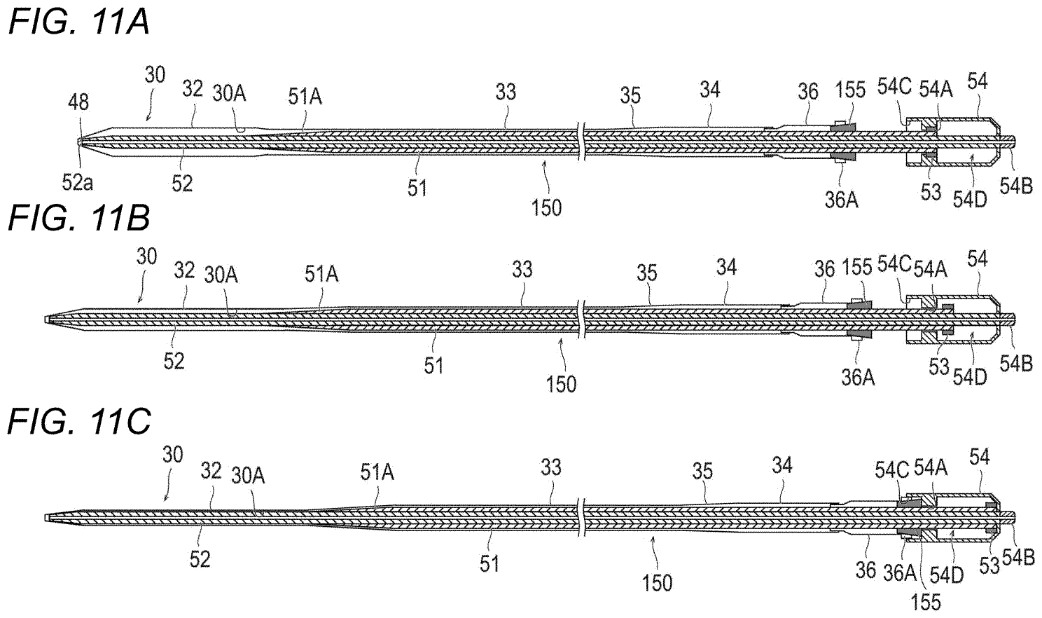

Next, a configuration of a stylet 150 according to a variation is described with reference to FIGS. 11A to 11C. Description of a part common to the configuration of the stylet 50 according to the embodiment described above is omitted. The stylet 150 according to the variation is different from the stylet 50 according to the embodiment described above in that a regulation member 155 is provided on an outer periphery of an outer peripheral member 51.

As illustrated in FIGS. 11A to 11C, the stylet 150 according to the variation includes the outer peripheral member 51 that extends in an axial direction, an inner peripheral member 52 provided on an inner periphery of the outer peripheral member 51, a fitting member 53 joined to an outer periphery of a proximal end of the outer peripheral member 51, a fitting stopper 54 into which the fitting member 53 may be fitted, and a regulation member 155 joined to the outer periphery of the outer peripheral member 51 spaced from a distal end side of the fitting member 53. Since configurations of the outer peripheral member 51, the inner peripheral member 52, the fitting member 53, and the fitting stopper 54 are the same as those of the stylet 50 according to the above-described embodiment, the description thereof is omitted.

The regulation member 155 is provided on the distal end side of the fitting member 53. The regulation member 155 is joined to the outer periphery of the outer peripheral member 51. A method of joining the regulation member 155 to the outer peripheral member 51 is not especially limited, and is, for example, adhesion using an adhesive.

An outer diameter of the regulation member 155 is formed to be slightly larger than an inner diameter of a male screw portion 36A so as to be fitted into an inner periphery of the male screw portion 36A of the lock connector 36. The regulation member 155 is formed of an elastically deformable material, and is fitted into the inner periphery of the male screw portion 36A of the lock connector 36 by elastic deformation of the regulation member 155. Note that, the regulation member 155 is not especially limited as long as this may regulate motion of the outer peripheral member 51 in the axial direction. For example, the regulation member 155 may be provided at a proximal end of the male screw portion 36A.

A distance from the proximal end of the regulation member 155 to a screw ring 54C of the fitting stopper 54 is the same as an extension length of the remaining expansion portion 32 when the shaft portion 33 starts contracting.

<Method of Using Stylet According to Variation>

Next, a method of using the stylet 150 according to the variation is described with reference to FIGS. 11A to 11C. FIGS. 11A to 11C are views for describing the method of using the stylet 150 according to the variation.

First, as illustrated in FIG. 11A, the stylet 150 is inserted into the lumen 30A of the catheter 30. The stylet 150 sequentially passes through the inside of the shaft portion 33 and the expansion portion 32, until a flat surface 52a of the inner peripheral member 52 of the stylet 150 abuts a receiving surface 48 of the distal end tip 41. At that time, the regulation member 155 becomes fitted into the inner periphery of the male screw portion 36A of the lock connector 36.

Then, the distal end of the expansion portion 32 is pulled toward the distal end side as illustrated in FIG. 11B. As a result, the catheter 30 receives a force to extend in the axial direction, and the expansion portion 32 having relatively high elasticity out of the catheter 30 extends in the axial direction. The motion of the outer peripheral member 51 in the axial direction may be regulated by the regulation member 155. Therefore, in this state, by moving the inner peripheral member 52 toward the distal end side with respect to the outer peripheral member 51, the expansion portion 32 of the catheter 30 extends in the axial direction and contracts radially inward. At that time, the fitting member 53 fitted into the fitted portion 54A of the fitting stopper 54 is displaced from the fitted portion 54A.

Thereafter, the stylet 50 is attached to the catheter 30 by screwing the female screw portion of the screw ring 54C into the male screw portion 36A provided on the lock connector 36 of the catheter as illustrated in FIG. 11C. At that time, as illustrated in FIG. 11C, proximal ends of the fitting member 53 and the outer peripheral member 51 abut a proximal end of a region 54D of the fitting stopper 54.

Following steps are similar to those in the method of using the stylet 50 according to the embodiment, so that the description thereof is omitted.

Although the catheter according to the present invention is described with the embodiment, the present invention is not limited only to the configuration described in the embodiment and variation, and may be appropriately changed based on the recitation in claims.

For example, in the above-described embodiment, the inner peripheral member 52 is formed of a material softer than that of the outer peripheral member 51, but the inner peripheral member 52 may be formed of a material as soft as that of the outer peripheral member 51.

The material forming the wire W is not limited to the shape memory material as long as the material has a restoring force to return to its original shape by deformation and has a function of reinforcing the resin layer; for example, the wire W may be formed of a known elastic material.

In the above-described embodiment, the outer diameter of the fitting member 53 is formed to be slightly larger than the inner diameter of the fitted portion 54A of the fitting stopper 54. However, there is no particular limitation as long as the fitting member is fittable into the fitted portion. For example, as illustrated in FIG. 12, an inner periphery of a fitting member 153 may be fitted so as to be engaged with an outer periphery of a fitted portion 154A.

What is claimed is:

1. A stylet configured to be inserted into a catheter tube provided with an expandable expansion portion, a shaft portion provided at a proximal end of the expansion portion, and a lumen through which blood may flow, the stylet comprising:
an outer peripheral member extending in an axial direction having an outer diameter conformable to an inner diameter of the shaft portion and having an inner passage defined by an inner periphery of the outer peripheral member;
an inner peripheral member received in the inner passage and provided with an exposed portion extending from a distal end of the outer peripheral member, wherein the inner peripheral member is slidable with respect to the outer peripheral member;
a fitting member joined to an outer periphery of the outer peripheral member at a proximal end of the outer peripheral member; and
a fitting stopper defining an inner region in which the outer peripheral member and the fitting member are movable over a length in the axial direction corresponding to an extension length of the expansion portion.

2. The stylet according to claim 1, wherein a length of the exposed portion in the axial direction corresponds to a length of the expansion portion before being extended.

3. The stylet according to claim 1, wherein a length in the axial direction of the region of the fitting stopper corresponds to a length by which the expansion portion extends in the axial direction when the shaft portion starts contracting.

4. The stylet according to claim 1, wherein a distal end part of the outer peripheral member is tapered toward the exposed portion of the inner peripheral member.

5. The stylet according to claim 1, wherein the inner peripheral member is formed of a material softer than a material of the outer peripheral member.

6. The stylet according to claim 1 further comprising:
a regulation member joined to an outer periphery of the outer peripheral member and configured to be fittable into a lock connector of the catheter tube.

7. A catheter system configured to be inserted into a living body to convey blood, comprising:
a catheter tube having an expandable expansion portion, a shaft portion provided at a proximal end of the expansion portion, and a lumen through which the blood may flow; and
a stylet configured to be inserted into the catheter tube, wherein the stylet comprises:
an outer peripheral member extending in an axial direction having an outer diameter conformable to an inner diameter of the shaft portion and having an inner passage defined by an inner periphery of the outer peripheral member;
an inner peripheral member received in the inner passage and provided with an exposed portion extending from a distal end of the outer peripheral member, wherein the inner peripheral member is slidable with respect to the outer peripheral member;
a fitting member joined to an outer periphery of the outer peripheral member at a proximal end of the outer peripheral member; and
a fitting stopper defining an inner region in which the outer peripheral member and the fitting member are movable over a length in the axial direction corresponding to an extension length of the expansion portion.

8. The catheter system according to claim 7, wherein a length of the exposed portion in the axial direction corresponds to a length of the expansion portion before being extended.

9. The catheter system according to claim 7, wherein a length in the axial direction of the region of the fitting stopper corresponds to a length by which the expansion portion extends in the axial direction when the shaft portion starts contracting.

10. The catheter system according to claim 7, wherein a distal end part of the outer peripheral member is tapered toward the exposed portion of the inner peripheral member.

11. The catheter system according to claim 7, wherein the inner peripheral member is formed of a material softer than a material of the outer peripheral member.

12. The catheter system according to claim 7 further comprising:
a regulation member joined to an outer periphery of the outer peripheral member and configured to be fittable into a lock connector of the catheter tube.
